(19) United States
(12) Patent Application Publication
(10) Pub. No.: US 2006/0148684 A1
(43) Pub. Date: Jul. 6, 2006

(54) Use of a histone deacetylase inhibitor for treating muscular dystrophies

(75) Inventors: Sabine De La Porte, Versailles (FR); Maurice Israel, Bures-sur-Yvette (FR); Vincent Voisin, Villemoisson (FR); Hafedh Haddad, Paris (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignee: Centre National de la Recherche Scientifique-CNRS, Paris (FR)

(21) Appl. No.: 10/536,417

(22) PCT Filed: Nov. 28, 2003
(86) PCT No.: PCT/FR03/03530

§ 371 Date: Sep. 27, 2005

(30) Foreign Application Priority Data
Nov. 28, 2002 (FR) 02/14980

(51) Int. Cl. A61K 38/12; A61K 31/473; A61K 31/19

(52) U.S. Cl. 514/9; 514/575; 514/290

(57) ABSTRACT

The invention relates to an inhibiter of histone deacetylase for treating or preventing a disease resulting from the deficiency of an adult gene in an individual by the re-expression of the homologous fetal gene. The invention concerns in particular the treatment of dystrophies such as Duchenne's dystrophy or Becker's dystrophy in which the defective adult gene is the dystrophin gene and the homologous fetal gene is the utrophin gene.

The present invention relates to the treatment of disease resulting from the deficiency of an adult gene in an individual due to the re-expression of the homologous fetal gene. The invention concerns in particular the long-term treatment of dystrophies and similar diseases and comprises the administration of an adequate quantity of an inhibitor of histone deacetylase to a subject presenting a defective gene.

The fetal metabolism is glycolitic and ammonotelic whereas the adult metabolism is oxidative and ureotelic. One of the principal compounds involved in the metabolism of the fetal type is butyrate, that associated with other products of the glycolitic metabolism, inhibits an essential enzyme at the nucleus level: Histone deacetylase. The release of the DNA strand that follows permits the transcription factors to reach the promoter and to induce the expression of the gene. Inversely, the stimulation of the oxidative metabolism associated with the stimulation of methylases induces the synthesis of new products hat replace those that were expressed during the fetal life. It is in this manner that the diminution of butyrate and lactate, by inducing the deacetylation of the histones, extinguishes the expression of the fetal genes whereas the methylation of metabolites, creatine phosphate, choline and others entrains the activation of the expression of the adult gene. The shift of the methylations toward the cytoplasm appears to be associated in a certain manner with the expression of an adult gene.

The transition between the expression of a fetal gene and that of the adult gene comes about because the metabolism becomes oxidative in order to adapt to air and to weight. This type of metabolism generates methylated transmitters and creatine phosphate, which permit the muscles, e.g., to adapt to life on the ground (in contrast to the fetal life, which takes place in an aquatic environment). The necessity of permuting fetal genes to adult genes probably corresponds to an adaptation of the organism to new sources of proteins.

Thus, the inventors observed that the fetal genes die out to the advantage of their adult homologues, or, more generally, that the genes less adapted to the environment of the organism die out to the advantage of homologues better adapted to this environment. These latter then express regulated proteins that respond in a more adequate manner to this environment.

Thus, a novel method of treating diseases in which the defective adult gene has a fetal homologue has been described in the prior art. This method is based on the reactivation of this fetal gene. It deals, e.g., with Duchenne's and Becker's myopathies or thalassemia and drepanocytosis. The French patent application published under No. 2 794 647 shows that the use of L-arginine and of NO donors permits the reactivation of the expression of fetal genes in adult tissues in such a manner as to restore the presence and the localization of fetal proteins. Spectacular effects were obtained in the case of muscular dystrophy in the mouse, in which the re-expression of utrophin significantly improves the state of the deficient muscles of the mdx mouse, which is a murine model of the human disease.

Furthermore, it has been reported that during its development the human fetus possesses a fetal hemoglobin with a strong affinity for oxygen, that will be replaced in the newborn by a low-affinity hemoglobin, which will be, in addition, regulated negatively by 2-3diphosphoglycerate (DPD). When the adult hemoglobin is defective on account of a mutation (falciform anemia), its regulatory ligand increases (Abekile, 1998).

The inventors now consider that this ligand can serve as an inducer signal for expressing fetal hemoglobin.

This mechanism of extinction-substitution depends on a “double switch”. The first one is general, non-specific and well-known and is connected to the state of the histones—it is known that their deacetylation, e.g., reduces the expression of genes by “compressing [tightening up, strengthening] the winding [coiling] of the DNA strand”. The second one is specific and would result from the decrease of an inductor that is no longer available when the adult gene or the best adapted one is expressed, because this inductor is then bound to the product of the specific gene. The existence of this specific switch has now been observed by experiments showing that the same “general switch”, inhibitor of histone deacetylase, will “ignite” fetal hemoglobin in falciform anemia, utrophin in Duchenne's dystrophy or SMN2 in spinal amyotrophy, etc.

Thus, products active on a general mechanism of expression are rendered permissive by the expression of the silent gene in each instance by the availability of the selective inductor NO or cGMP for utrophin, 2-3DPG or derivative for fetal hemoglobin, Ch3SM, Ch3SmRNP or derivative for SMN2. This ligand, that no longer finds its specific target on account of the mutation, then authorizes the action of the general switch, that preferentially ignites the silent gene corresponding to the mutation.

It is then sufficient that the mutation makes the ligand of a product of the mutated selective gene available in each instance in order that the inhibitor of histone deacetylase can preferentially ensure the expression of the homologous gene of the mutated gene. This has multiple applications because each time a product has been active in a given mutation by acting on the general switch and has permitted the expression of the silent gene, it can be foreseen that it will be active in all the other pathologies.

Research carried out within the framework of the invention has confirmed that this regulation is only released if a general inhibition of the silent genes is lifted [removed], which is connected to the acetylation state of the histones (Zang and Reinberg, 2001).

The inhibitors of histone deacetylase favoring the expression of fetal genes are nonspecific. Under these conditions the fetal gene corresponding to the adult mutated gene is specifically activated (the fetal hemoglobin in the case of falciform anemia, or utrophin in the case of Duchenne's dystrophy) by virtue of the existence of another switch specific for each couple of fetal-adult genes.

The mutation of the adult protein is indicated and releases the activation of the fetal protein. The adult proteins adapted to the partial pressure of the oxygen in the air and to the weight for the muscular proteins are regulated by specific ligands. A typical example is 2-3DPG, that regulates adult hemoglobin. When the adult protein is absent or mutated its specific ligand is then found free in the cytoplasm and by virtue of this fact directly or indirectly induces the activation of the corresponding fetal gene. This induction is possible only in the instance in which histone deacetylase is inhibited, e.g., by butyrate, that is, in the situation of a glycolitic metabolism, which is the case for juvenile cells.

It has in fact been determined that in its hypoacetylated form histone brakes the expression of these genes. In the presence of butyrate or of other inhibiters of histone deacetylase this general inhibition is lifted and the selective inductor, activated by virtue of the mutation, is then permitted to release the silent gene. The inhibitors of histone deacetylase such as butyrate are, for the rest, used in the treatment of falciform anemia.

This same principle can also be applied to spinal amyotrophy and with SMN1 being mutated its CH3-SmRNP (methyl, small ribonucleoprotein) ligand would become inductive if, however, the butyrate permits it to act favoring the re-acetylation of the histones. The expression of SMN2 was thus obtained by butyrate (Chang et al., 2001). It would also be useful in this instance to favor the methylation of SmRNP or to augment its expression. The NO donors could be useful as well as the donors of methyl. The present invention can also be applied to Miyoshi myopathy (MM) and to girdle myopathy or form 2B of limb girdle muscular dystrophy (LGMD2B) (K. Bushby, Acta Myologica, vol. 19, 2000, pp. 209-13), that are characterized by the absence of dysferlin, a homologous protein, and myoferlin can then serve as a substitute (Davis et al., Hum., Mol. Genet., 2000, vol. 9, pp. 217-226). It can also concern a myasthenic syndrome called gamma-AchR (receptor of acetylcholine) in which the gamma subunit (fetal form) of the receptor for acetylcholine is not replaced by the epsilon subunit (adult form) (Engel et al., Ann. Neurol. 1996, vol. 40, pp. 810-817). For these two types of pathologies, in conformity with the invention the general ligand is an inhibitor of histone deacetylase (HDAC) and the specific ligand is a phospholipid for MM and LGMD2B and choline for the gamma-Achr syndrome. In fact, dysferlin and myoferlin form part of the family of molecules in domain C2 that recognizes and strongly bonds phospholipids.

The use of a compound based on butyrate for treating beta hemoglobinopathies, falciform anemia and symptoms of beta thalassemia has been reported in the prior art (S. P. Perrine et al., EXPERIENTIAL, BIRKHAUSER VERLAG, BASEL, CH, vol. 49, No. 2, Feb. 15, 1993 (2/15/1993), pp. 133-137). It is appropriate to remark that this document is not interested in muscular dystrophies such as Duchenne's dystrophy and that it proposes arginine butyrate as a derivative of butyrate in order to avoid a sodium overload in the patient. Thus, the active principle which these authors propose using is butyrate and not arginine.

The extension of a principle used for treating falciform anemia and beta thalassemia to another monogenic pathology, Duchenne's muscular dystrophy, has also been envisaged in the prior art (N. F. Olivieri et al., HUMAN MOLECULAR GENETICS 1998 UNITED KINGDOM, vol. 7, No. 10, 1998, pp. 1655-1658, XP002249547, ISSN: 0964-6906). This article is based on the hypothesis of a principle common to these two types of pathologies, the pharmacological stimulation of a fetal gene for replacing an adult gene (fetal hemoglobin in one instance and utrophin, that is the fetal homologue of the dystrophin absent in dystrophic patients in the other instance).

The present invention is based on the implementation of a double switch system for lifting the inhibition of the fetal gene: i) the general switch connected to the acetylation of histones, and ii) the selective switch connected to the product of the single lacking gene (NO in the case of utrophin). Thus, the inhibiter of histone deacetylase opens the general switch and arginine (NO) opens the selective switch.

The inventors have thus now observed that a switch for histone deacetylase could be used for the preparation of a drug for the treatment or prevention of a disease resulting from the deficiency of an adult gene in an individual by the re-expression of the homologous fetal gene. This drug in accordance with the invention is intended to reactivate the expression of at least one fetal gene in adult tissues in such a manner as to restore the presence and/or the localization of at least one fetal protein. Thus, the invention aims to reactivate the fetal gene coding the embryonic form of the protein coded by the defective adult gene.

The invention is particularly concerned with the treatment of muscular dystrophies such as the Duchenne's or Becker's dystrophy, in which the defective adult gene is the dystrophin gene and the homologous fetal gene is the utrophin gene.

In fact, it was observed that dystrophin is spatially very close to the NO synthase and the locally produced NO will doubtless have essential effects on this protein, whose mutation is accompanied by a deficit of NO synthase on the membrane.

Thus, the NO permits the induction of the expression of utrophin, the silent homologue of dystrophin, that predominates in the fetal life and which, for the rest, only subsists in the adult in locations where the NO synthase is very elevated (motor end-plate, vessels).

Thus, the inventors have demonstrated that arginine, substrate of NO synthase, and the donors of NO bring about an overexpression of utrophin. This effect was obtained without lifting the general switch connected to the acetylation of histones because the NO also blocks the essential steps of the Krebs cycle, which brings about an elevation of acetylCoA and of the cetonic bodies (butyrate). Thus, NO acts as an inductive ligand but also via the butyrate on the deacetylase histone.

The implementation of an inhibiter of histone deacetylase in accordance with the invention also offers the advantage of having a drug that can be administered orally.

The action of L-arginine and its derivatives on the reactivation of the fetal gene is obtained parenterally. It is difficult to apply such a therapy over the long term and it is necessary to provide periods of rest. The inhibiters of histone deacetylases advantageously administered orally permit the effect to be maintained during these periods. In fact, the inhibiters of histone deacetylases permit the “general switch” connected to the acetylation of histones to be kept open during these periods.

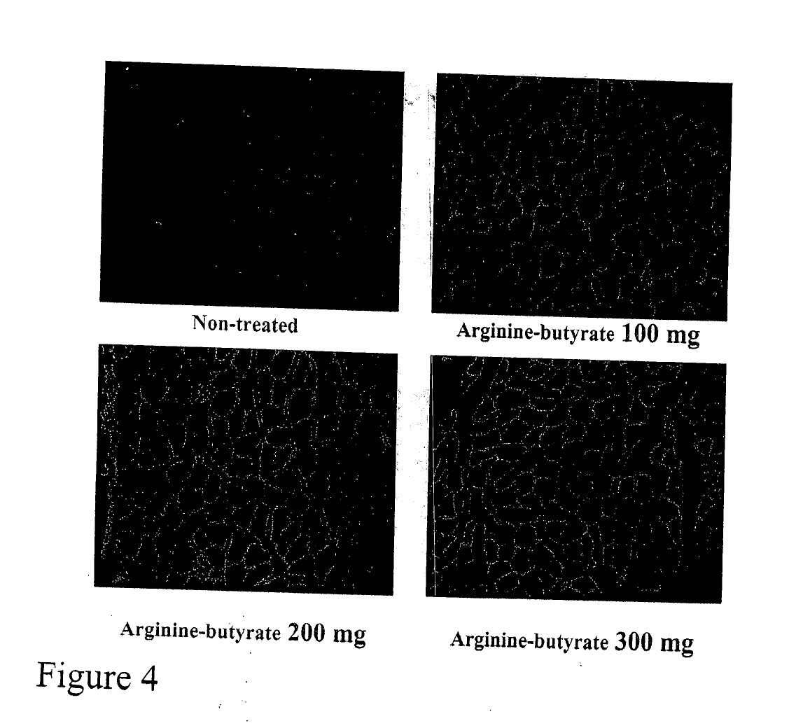

There are numerous inhibiters of histone deacetylase: Valproate, trichostatin, etc., that maintain the histones acetylated, of which the most classic is butyrate. By virtue of the fact that dystrophin and utrophin are associated with NO synthase, the invention have demonstrated that NO favors the expression of utrophin according to the same principle as 2-3-DPG in the case of hemoglobin. The compound principle used for inducing the expression of utrophin is the substrate of NO synthase: L-arginine. The inventors have now shown (FIG. 4) that a salt of arginine butyrate significantly augments the quantity of utrophin in the healthy muscles and in the mdx mouse.

The inhibiter of histone deacetylase can thus be selected from the group comprising butyrate, phenylbutyrate, isobutyramide, valproate, the hydroxamate derivative of butyric acid, apicidin, CBHA (m-carboxycinnamic acid bishydroxyamide), HC toxin, M344 (4-dimethylamino-N-(6-hydroxycarbamoyl-hexyl)benzamide), Nullscript (4-(1,3-dioxo-1H,3H-benzo[de]isoquinolin-2-yl)-N-hydroxybutanamide), SAHA (suberoylanilide hydroxamic acid), Scriptaid (6-(1.3-dioxo-1H,3H-benzo[de]isoquinolin-2-yl)-N-hydroxyhexanamide), trichostatin (TSA; (R-(E,E)-7-[4-(dimethylamino)-phenyl]-N-hydroxy-4,6-dimethyl-7-oxo-2,4-heptadienamide).

Numerous genetic diseases have as their origin a mutation on a gene that has a silent copy. The present invention therefore has the problem of re-expressing this silent copy using an entire series of a combination of compounds of the type previously described.

A particular embodiment of the invention relates to bifunctional compounds in which the ligand of the absent or abnormal protein is bound by covalence to an inhibitor of histone deacetylase. Such compounds have the ability to treat pathologies in which a gene is mutated by inducing the expression of a homologous gene, primarily the fetal copy of this gene (in the instance that the latter exists), using a cleavable bifunctional compound that releases in the organism i) the inhibitor of histone deacetylase acting on the general, non-specific switch connected to the acetylation of histones, and ii) a ligand of the mutated protein activating the specific switch. In the instance in which this ligand is not available or would be unusable, it is possible, as a replacement, to connect its precursor or its product or also a molecule favoring its action.

Therefore, the invention is particularly concerned with the use in association of an inhibitor of histone deacetylase and of a compound regulating a protein coded by an adult gene for the preparation of a drug intended for the treatment or the prevention of a disease resulting from the deficiency of this adult gene for which a silent homologous gene exists.

In a particular embodiment this compound is suitable for bonding to and regulating the protein coded by this adult gene. This can be, e.g., allosteric proteins.

According to a first implementation of the invention this drug contains the inhibiter of histone deacetylase and the compound regulating a protein coded by an adult gene in a separated manner in the same package.

According to a second implementation of the invention this drug contains the inhibiter of histone deacetylase and the compound regulating a protein coded by an adult gene in a single pharmaceutical form containing the two ingredients.

According to a third implementation of the invention this drug contains the inhibiter of histone deacetylase and the compound regulating a protein coded by an adult gene are bound by covalence, possibly by the intermediation of a spacer arm. This bond or spacer arm is cleavable in the organism in such a manner as to free the two active ingredients.

According to a first specific application of the invention the inhibiter of histone deacetylase is associated with at least one compound selected from the group comprising NO, a donor compound of NO or a compound capable of freeing, favoring or inducing the formation of NO in the cells. Said drug is intended for the treatment or prevention of a disease resulting from the deficiency of the dystrophin gene by permitting the re-expression of the utrophin gene. This disease is Duchenne's or Becker's dystrophy.

A drug comprising histone deacetylase is administered to an individual having received concomitantly with or previously to the administration of this drug an appropriate quantity of at least one compound selected from the group comprising NO, a donor compound of NO or a compound capable of freeing, favoring or inducing the formation of NO in the cells.

The compound capable of inducing the formation of NO is, e.g., L-arginine or one of its derivatives constituting a substrate of NO synthase or favoring the availability of the substrate. Thus, a preferred example of the first implementation of the invention below concerns a pharmaceutical composition comprising arginine butyrate.

The donor compound of NO is, e.g., molsidomine or one of its derivatives capable during their transformation into the organism of releasing NO.

According to a specific second application the invention relates to the treatment of spinal amyotrophy in which the inhibitor of histone deacetylase is associated with a donor compound of methyl capable of activating endogenous SmRNP (small ribonucleoprotein) by methylating it. CH3-SmRNP is capable of bonding the proteins coded by the SMN1 gene, and when the latter is deficient, it permits the re-expression of the homologous gene SMN2 by associating with the inhibitor of histone deacetylase.

According to a specific third application of the invention the inhibitor of histone deacetylase is associated with one or several phospholipids capable of bonding to dysferlin. This drug is intended for the treatment or the prevention of a disease resulting from the deficiency of the dysferlin chain by permitting the pre-expression of myoferlin. This disease is the Miyoshi myopathy (MM) or the 2B form of limb girdle muscular dystrophy (LGMD2B).

According to a specific fourth application of the invention the inhibitor of histone deacetylase is associated with choline or a derivative of the latter capable of bonding the nicotinic receptor acetylcholine. This drug is intended for the treatment or the prevention of a disease resulting from the deficiency of the gene of the epsilon subunit of said adult receptor by permitting the re-expression of the homologous fetal gene coding the gamma subunit of this receptor. This disease is a myasthenic syndrome called gamma-AchR.

According to a specific fifth application of the invention the inhibitor of histone deacetylase is associated with 2-3-diphosphoglycerate, a derivative or precursor (inosine) of the latter capable of fixing itself onto hemoglobin. This drug is intended for the treatment of falciform anemia by permitting the re-expression of fetal hemoglobin.

As previously indicted, the invention aims to offer novel bifunctional products in which the inhibiter of histone deacetylase and the compound capable of bonding to a protein coded by an adult gene are bonded by covalence, possibly by the intermediation of a spacer arm.

The two ingredients are advantageously connected by ester or amide bonds. The chemical groups ensuring the covalent bond are, e.g., carboxylic ester, carboxylic amide, thiocarboxylic ester or thiocarboxylic amide groups.

In the case of falciform anemia the bifunctional product connects by an ester or amide bond, an inhibiter of histone deacetylase (valproate, butyrate or other) to the allosteric ligand of adult hemoglobin such as 2-3-diphosphoglycerate (DPG). It is also possible to replace the 2-3-DPG by its precursor, inosine. Once administered to the patient the compound is cleaved by the esterases or amidases at the level of the ester or amide bond (these enzymes are abundant in the cells and in the blood). The released inhibiter of histone deacetylase will act in a non-specific manner on the general switch as a function of the histones, whereas the 2-3-DPG or its precursor will specifically activate the fetal hemoglobin switch. In other words, since the histones are acetylated, in principle, any gene could be activated, but 2-3-DPG will specifically activate the gene of fetal hemoglobin because it will not have found its target, adult hemoglobin.

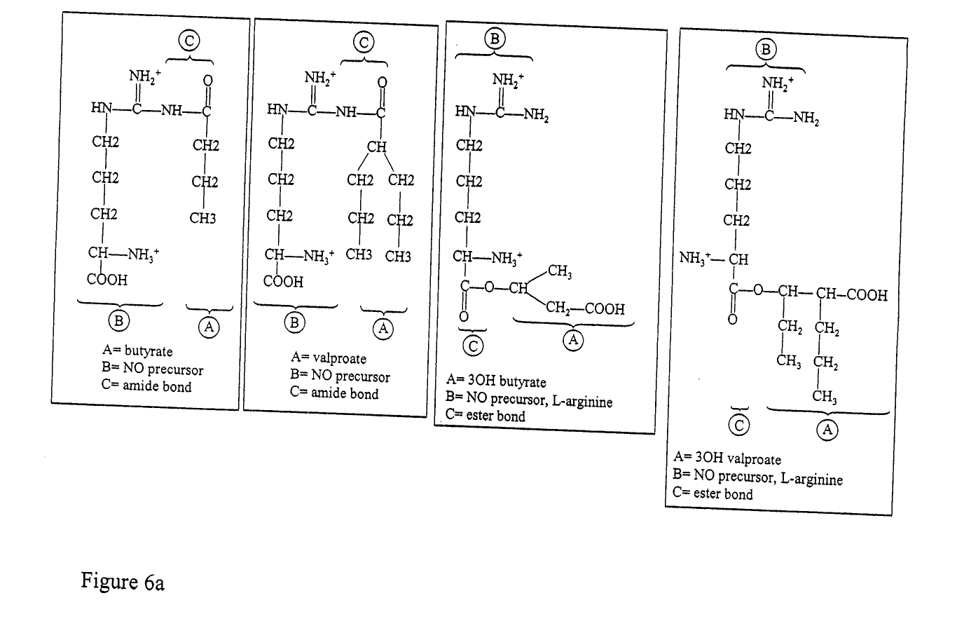

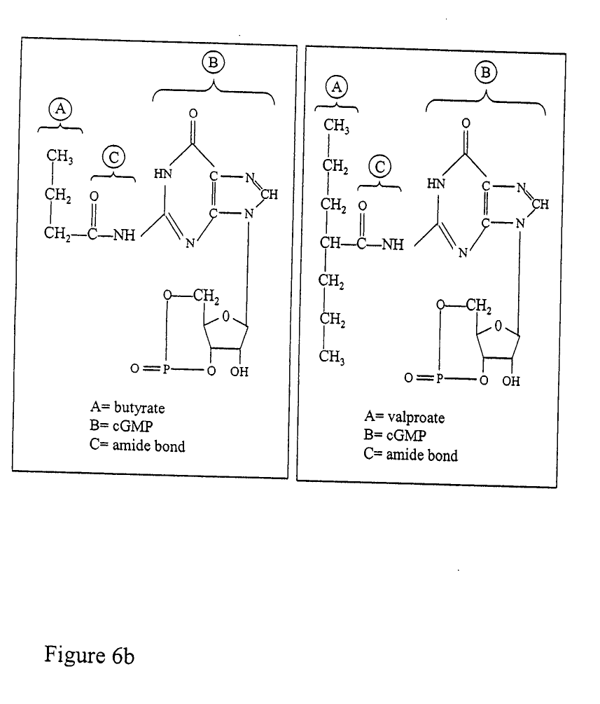

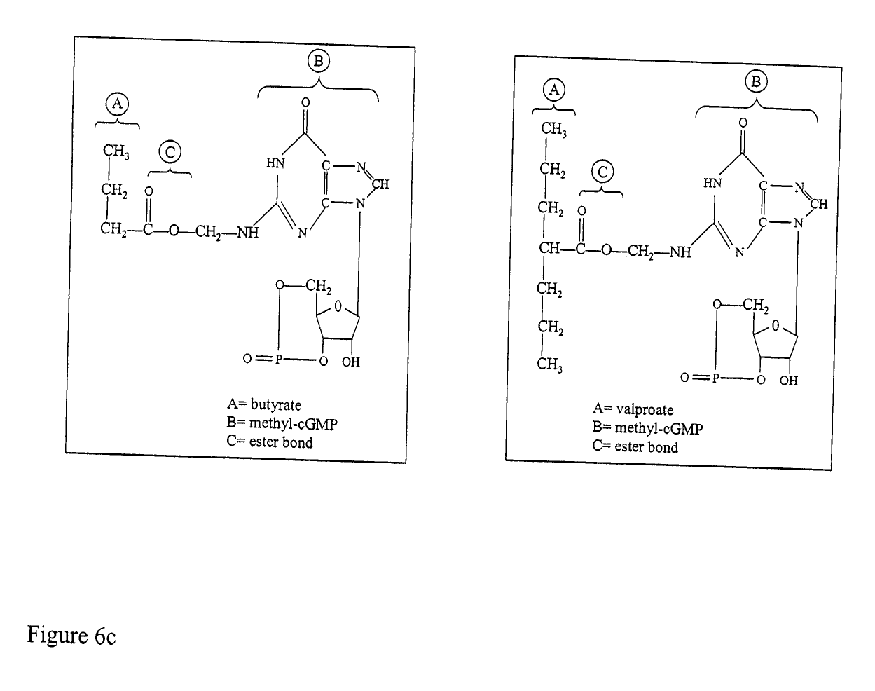

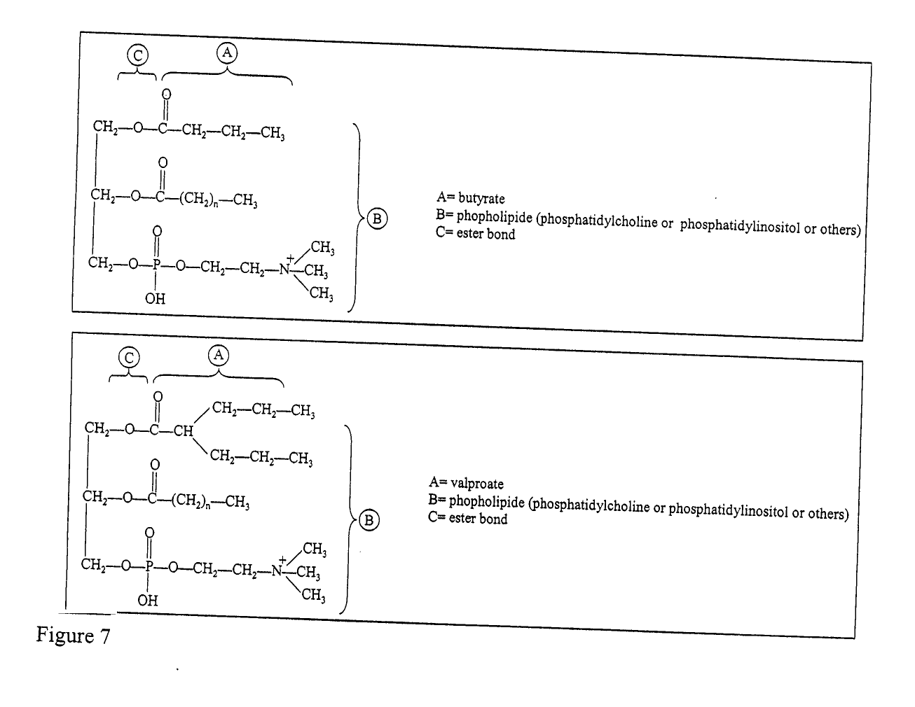

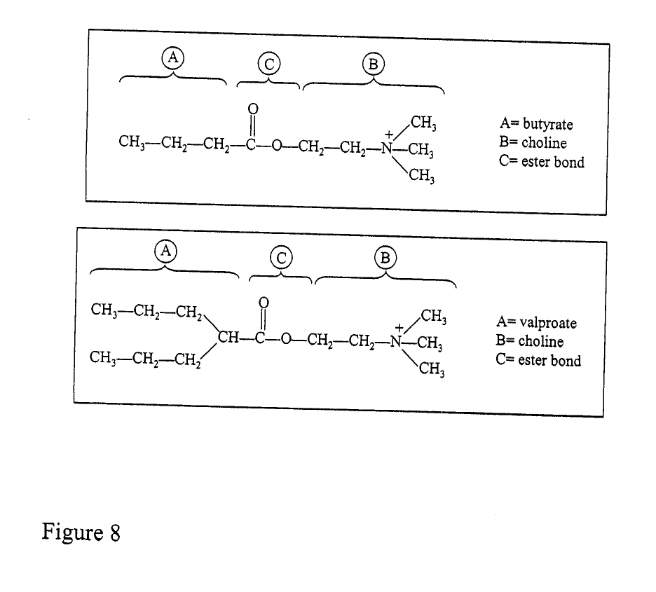

The same principle can be applied to induce utrophin. FIG. 6 shows a covalent amide bond connecting valproic or butyric acid to the arginine amine (which is different from a salt obtained by neutralization). The cleavage of the amide bond by amidase in the cells will release the inhibitor of histone deacetylase and the precursor of NO. Another possibility, described in FIG. 6a, is to form an ester bond between the 30H butyrate or the OH valproate and the carboxyl group of the arginine. In each case the main switch dependent on histone is activated whereas the signal NO that is generated will specifically induce the expression of utrophin. Instead of L-arginine, precursor of NO, it is possible to use the cGMP bound to the butyrate or to the valproate by an amine or ester bond (FIG. 6). In the case of Miyoshi myopathy or of the form of girdle myopathy in which dysferlin is mutated (LGMD2B), the latter can be replaced by myoferlin. In this case the compound to be synthesized connects the inhibitor of histone deacetylase (butyrate or valproate or others) by an ester bond to the dysferlin ligand. The dysferlin ligand is probably a phospholipid because dysferlin comprises a domain C2 common to other proteins, a domain which bonds phospholipids. FIG. 7 shows an example where the ester bond could again be exchanged by an amine bond. Another example concerns the congenital syndromes of myasthenia due to a mutation of the adult ε subunit of the receptor for which it would be necessary to reactivate the expression of the γ subunit that confers its fetal form to the nicotinic receptor. The bifunctional compound proposed in this instance would associate the inhibiter of histone deacetylase (butyrate or valproate or others) via an ester bond, for example, to a specific ligand of the receptor such as choline (FIG. 8). The cleavage of the ester bond by esterases releases the butyrate or the valproate, opening the general switch and also releasing the specific inductor of the fetal form of the nicotinic receptor, choline.

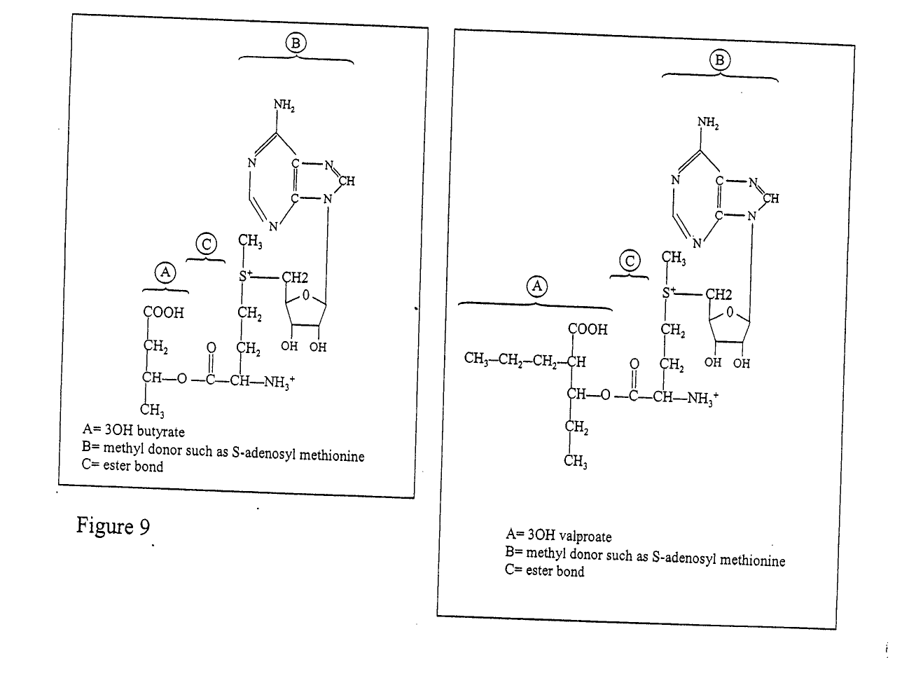

Another case is that of spinal amyotrophy—the ligand of the SMN1 protein could be directly or indirectly snRNPs, that is involved in the splicing of mRNA. The methylation of the snRNP complex appears necessary for its functioning. It would be dangerous to use snRNPs as specific inductor (the antibodies directed against snRNPs cause Lupus erythematosus). In this situation it is only possible to aid the functioning of snRNPs by favoring their methylation. A methyl donor could be connected by an ester or amine bond to an inhibiter of histone deacetylase (butyrate, valproate), as we previously described. This compound (FIG. 9) would act, after cleavage, on the general switch and on the specific induction of the ligand formed by the methylation of snRNPs. The objective is to induce the expression of SMN2, the silent copy of the SMN1 gene, including its exon 7.

The invention also relates to pharmaceutical compositions comprising at least one product as previously defined by way of active agent.

Other advantages and characteristics of the invention will appear from the following examples in which the attached drawings are referred to.

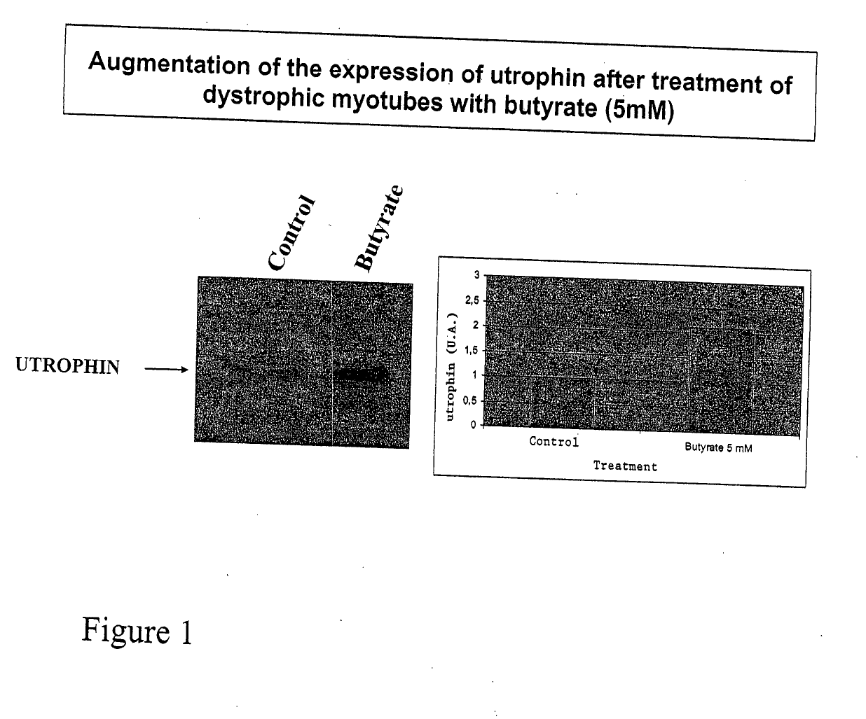

FIG. 1 illustrates the possibility of inducing an augmentation of utrophin in the muscle under the effect of inhibitors of histone deacetylase using butyrate, at the top of the list of these inhibitors on cultures of murine myotubes.

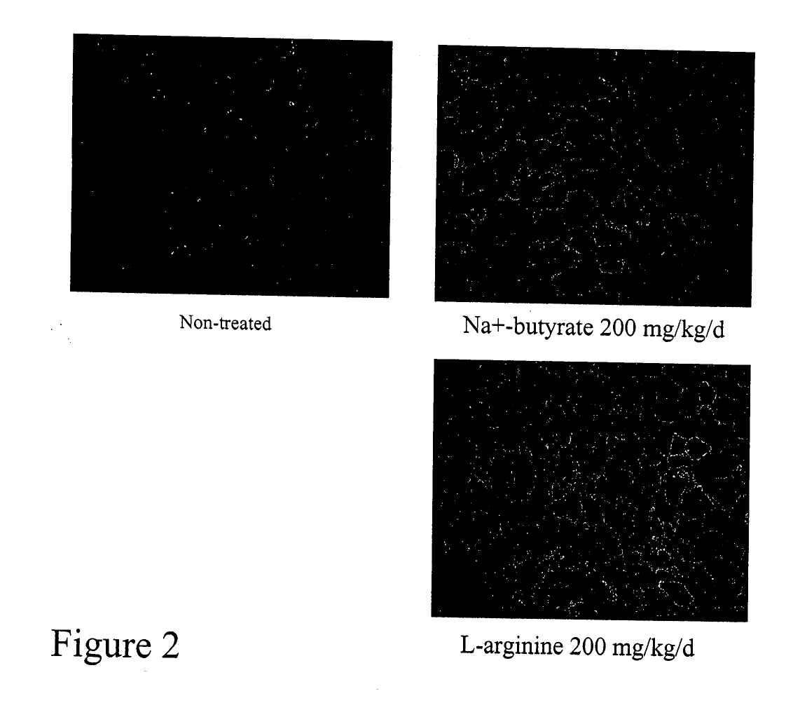

FIG. 2 shows the analysis by immunofluorescence of the utrophin present in the muscles of dystrophic mdx mice, the animal model of Duchenne's myopathy, which were injected with butyrate and for the sake of comparison with L-arginine as NO donor.

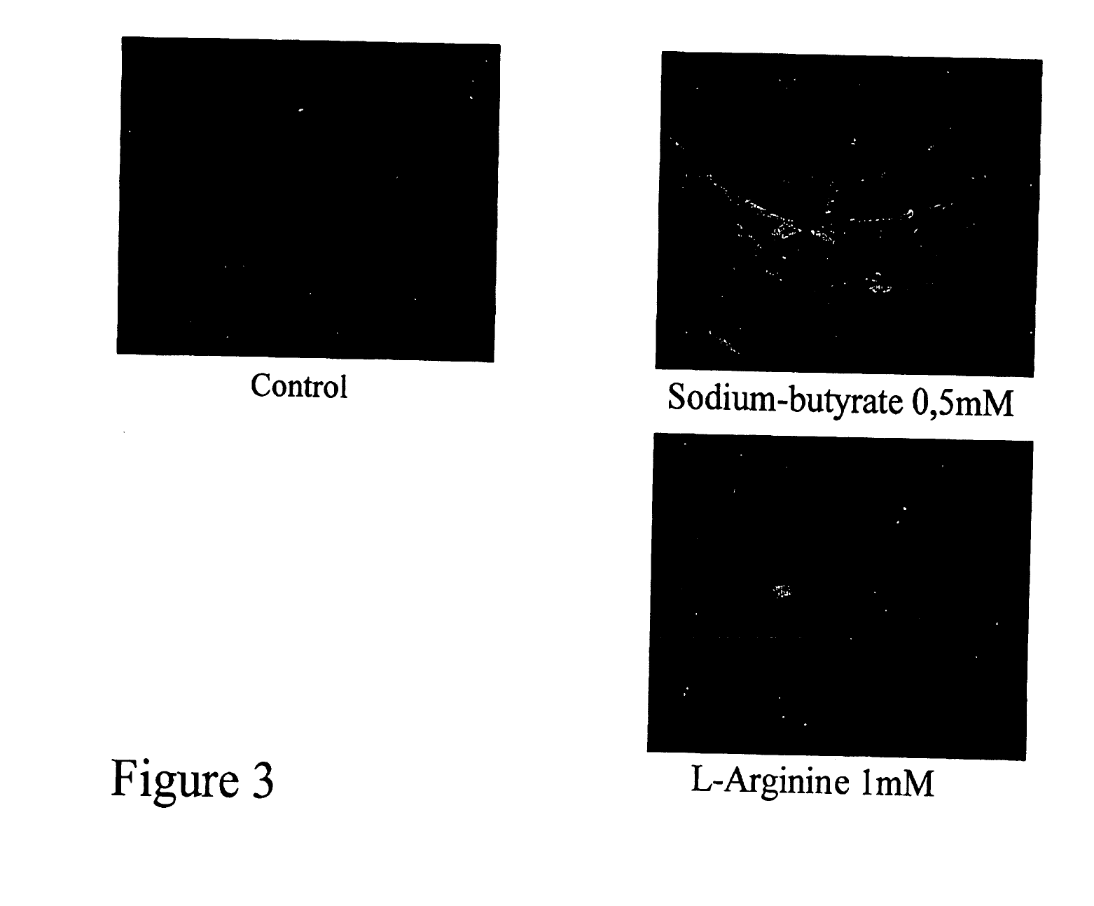

FIG. 3 shows the analysis by immunofluorescence of utrophin after application of the same molecules as for FIG. 2 to human myotubes in culture.

FIG. 4 shows the augmentation of utrophin in healthy mice treated with arginine butyrate.

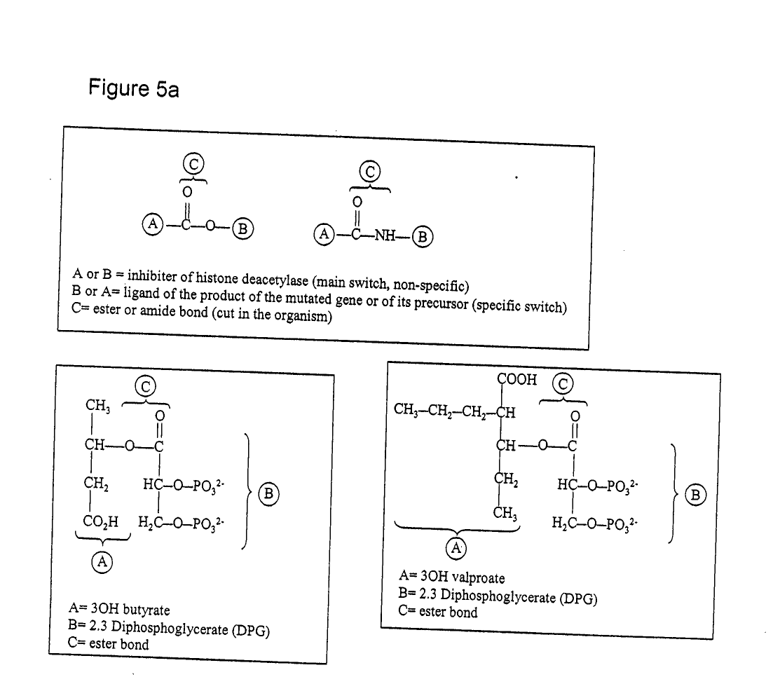

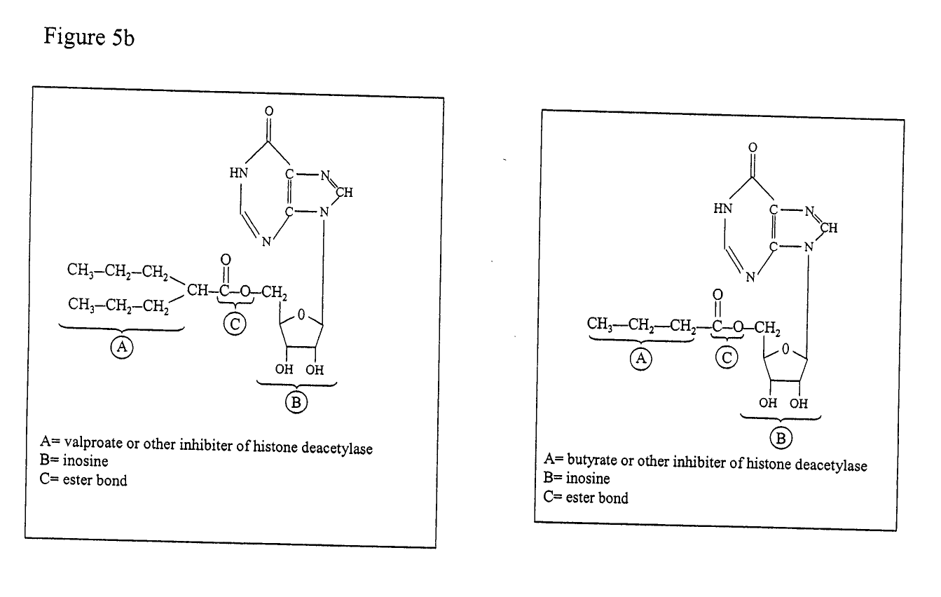

FIG. 5 shows examples of bifunctional products that can be used in the case of falciform anemia.

FIG. 6 shows examples of bifunctional products that can be used in the case of Duchenne's muscular dystrophy.

FIG. 7 shows examples of bifunctional products that can be used in the case of Miyoshi myopathy and of LGMD2B.

FIG. 8 shows examples of bifunctional products that can be used in the case of congenital myasthenic syndrome (mutation of the epsilon subunit of the nicotinic receptor.

FIG. 9 shows examples of bifunctional products that can be used in the case of spinal amyotrophy.

### EXAMPLE 1

### Augmentation of Utrophin in the Myotubes of Mdx Mice Treated with Butyrate (FIG. 1)

Myotubes of dystrophic mdx mice (line xlt) are treated with 5 mM of butyrate for 48 h. The augmentation of utrophin is then quantified in Western blot. The measuring of the intensity of the bands shows an augmentation of expression of utrophin by a factor of 2 after treating the myotubes with butyrate.

### Example 2

### Augmentation of Utrophin in Mdx Mice Treated with Butyrate (FIG. 2)

Mdx mice were injected daily i.p. and for 6 weeks with 200 mg/kg/day either with butyrate or with L-arginine or with physiological serum. It is observed that the utrophin is poorly expressed in the muscle of the mdx mice injected with physiological serum used as control. On the other hand, in the mice injected with butyrate or L-arginine the utrophin appears under the muscular membrane. It should be noted that the augmentation of utrophin is greater in the case of the mice injected with butyrate. The augmentation of the utrophin was quantified in Western blot. The measuring of the intensity of the bands shows an augmentation of expression of utrophin by a factor of 2 in the mdx mice treated with butyrate.

### Example 3

### Augmentation of Utrophin in Human Myotubes Treated with Butyrate (FIG. 3)

Human myotubes were obtained after placing operational residues supplied by the BTR (Tissue Bank for Research) in culture. These myotubes, once differentiated, were treated with butyrate or L-arginine. As in the case of injected mice, the augmentation of utrophin visualized by immunofluorescence is particularly significant after a treatment with 0.5 mM butyrate.

### Example 4

### Augmentation of Utrophin in Healthy Mice Treated with Arginine Butyrate (FIG. 4)

OF1 mice were injected daily with i.p. and for 6 weeks with 100, 200 or 300 mg/kg/day either with arginine butyrate or with physiological serum. No utrophin was observed on the membrane in the muscle of the mice injected with the physiological serum used as control (“non-treated”). On the other hand, in the mice injected with arginine butyrate, utrophin appears very clearly under the muscular membrane. This augmentation is dosage-dependent. The induction of utrophin under the muscular membrane is significant probably on account of an additive effect of the arginine via the NO and of the butyrate via the activation of the transcription of the utrophin gene.

### BIBLIOGRAPHIC REFERENCES

Adekile, 1998. Ann. Trop. Paediatr. 9: 165-168.
Chang et al., 2001. Proc. Natl. Acad. Sci. 98: 9808-9813.
Chaubourt et al., 1999. Neurobiol. Diseases 6: 499-507.
Chaubourt et al., 2000. CR. Acad. Sci. III-Vie 323, 735-740.
Chaubourt et al., 2001. J. Physiol. (Paris) 96, 43-52.
Chaubourt E., Fossier P., Baux G., Leprince C., Israël M., De La Porte S., (1999). Patent No. 99/07442, Pharmaceutical composition comprising NO or at least a compound capable of releasing or inducing the formation of NO in the cells.
Zang and Reinberg, 2001. Genes Dev. 15: 2343-2360.

1-31. (canceled)

32. A method of treating or preventing a disease resulting from a deficiency of an adult gene for which there is a silent homologous gene comprising administering to a patient a therapeutically effective amount of an inhibitor of histone deacetylase that permits re-expression of the homologous gene.

33. The method according to claim 32, wherein the homologous gene is the fetal gene.

34. The method according to claim 32, wherein the inhibitor reactivates expression of at least one fetal gene in adult tissues to restore the presence and/or localization of at least one fetal protein.

35. The method according to claim 34, wherein the fetal gene codes an embryonic form of the protein coded by the defective adult gene.

36. The method according to claim 32, wherein the inhibitor of histone deacetylase is selected from the group consisting of butyrate, phenylbutyrate, isobutyramide, valproate, the hydroxamate derivative of butyric acid, apicidin, CBHA (m-carboxycinnamic acid bishydroxyamide), HC toxin, M344 (4-dimethylamino-N-(6-hydroxycarbamoyl-hexyl) benzamide), Nulscript (4-(1,3-dioxo-1H,3H-benzo[de]isoquinolin-2-yl)-N-hydroxybutanamide), SAHA (suberoylanilide hydroxamic acid), Scriptaid (6-(1.3-dioxo-1H,3H-benzo[de]isoquinolin-2-yl)-N-hydroxyhexanamide), trichostatin (TSA; and (R-(E,E)-7-[4-(dimethylamino)-phenyl]-N-hydroxy-4,6-dimethyl-7-oxo-2,4-heptadienamide).

37. The method according to claim 32, wherein the inhibitor is orally administered.

38. The method according to claim 32, wherein the disease is selected from the group consisting of dystrophies comprising Duchenne's or Becker's dystrophies, falciform anemia, myopathies comprising Miyoshi myopathy and LGMD2B, congenital myasthenic syndrome and spinal amyotrophy.

39. The method according to claim 38, wherein the disease is Duchenne's dystrophy or Becker's dystrophy and the defective adult gene is the dystrophin gene and the homologous fetal gene is the utrophin gene.

40. The method according to claim 38, wherein the disease is spinal amyotrophy and the defective adult gene is the SMN1 gene and the homologous gene is the SMN2 gene.

41. The method according to claim 38, wherein the disease is Miyoshi myopathy or form 2B of girdle myopathy and the defective adult gene is the dysferlin gene and the homologous gene is the myoferlin gene.

42. The method according to claim 38, wherein the disease is a myasthenic syndrome (gamma-AchR), and the defective adult gene is the gene of the epsilon subunit of the nicotinic receptor on acetylcholine and the homologous gene is the gene of the gamma subunit of this receptor.

43. The method according to claim 38, wherein the disease is falciform anemia, and the defective adult gene is the hemoglobin gene and the fetal homologous gene is the fetal hemoglobin gene.

44. A method of treating or preventing a disease resulting from a deficiency of an adult gene for which there is a silent homologous gene comprising administering to a patient a therapeutically effective amount of an inhibitor of histone deacetylase and a compound regulating a protein coded by the adult gene.

45. The method according to claim 44, wherein the compound bonds and/or regulates the protein coded by the adult gene.

46. The method according to claim 44, wherein the inhibitor and the compound are separated, but are in the same package.

47. The method according to claim 44, wherein the inhibitor and the compound are in a single pharmaceutical form containing the two active ingredients.

48. The method according to claim 47, wherein the inhibitor and the compound are connected by covalence, optionally by a spacer arm, and the bond or spacer arm is cleavable in an organism to release the two active ingredients.

49. The method according to claim 44, wherein the disease is a dystrophy resulting from deficiency of a dystrophin gene by permitting re-expression of a utrophin gene, and the inhibitor is associated with at least one compound selected from the group consisting of NO, an NO donor compound or a compound that releases, favors or induces formation of NO in cells.

50. The method according to claim 44, wherein the disease results from a deficiency of an adult gene by re-expression of a homologous fetal gene, and the patient received concomitantly with or previously to administration of the inhibitor a therapeutically effective amount of at least one compound selected from the group consisting of NO, a donor compound of NO or a compound that frees, favors or induces formation of NO in cells.

51. The method according to claim 49 or 50, wherein the compound that induces formation of NO is L-arginine or a derivative constituting a substrate of 0 synthase or favoring availability of the substrate.

52. The method according to claim 49 or 50, wherein the donor compound of NO is molsidomine or a derivative that releases NO during transformation into the patient.

53. The method according to claim 44, wherein the disease is spinal amyotrophy, the inhibitor is associated with a methyl donor compound that activates endogenous SmRNP by methylation, and CH3-SmRNP that bonds a protein coded by the SMN1 gene, and when the SMN1 gene is deficient, permitting re-expression of homologous gene SMN2.

54. The method according to claim 44, wherein the disease is Miyoshi myopathy or form 2B of girdle myopathy and the inhibit is associated with one or several phospholipids that bonds dysferlin, and when the dysferlin is deficient, permitting re-expression of myoferlin.

55. The method according to claim 44, wherein the disease is a myasthenic syndrome (gamma-AchR), and the inhibitor is associated with choline or a derivative that bonds a nicotinic receptor to acetylcholine, and when a gene of a epsilon subunit is deficient, permitting re-expression of a homologous fetal gene coding a gamma subunit of the receptor.

56. The method according to claim 44, wherein the disease is falciform anemia, the inhibitor is associated with 2-3-diphosphoglycerate, a derivative or a precursor that fixes onto hemoglobin, and, when the hemoglobin is deficient, permitting re-expression of fetal hemoglobin.

57. A product in which an inhibitor of histone deacetylase and a compound regulating a protein coded by an adult gene are connected by covalance, optionally with a spacer arm, which connection or spacer arm is cleavable in an organism in such a manner as to release two active ingredients.

58. The product according to claim 57, wherein the compound bonds and/or regulates the protein connected by the adult gene.

59. The product according to claim 57, wherein the two ingredients are connected by ester or amide bonds.

60. The product according to claim 57, wherein chemical groups ensuring the covalence are carboxylic ester, carboxylic amide, thiocarboxylic ester or thiocarboxylic amide groups.

61. The product according to claim 57, wherein the inhibitor of histone deacetylase and the compound regulating a protein coded by an adult gene.

62. A pharmaceutical composition conmprising at least one product in accordance with claim 57.
